(19) United States
(12) Patent Application Publication
(10) Pub. No.: US 2008/0318330 A1
(43) Pub. Date: Dec. 25, 2008

(54) Method of standardization of bacterial polysaccharides found in rumen fluid

(76) Inventor: Robert Terrell Winn, Big Sandy, TX (US)

Correspondence Address: Robert Terrell Winn, 141 PR 3808, Big Sandy, TX, 75755, US

(21) Appl. No.: 12/217,542

(22) Filed: Jul. 8, 2008

Related U.S. Application Data
(62) Division of application No. 10/923,313, filed on Aug. 23, 2004.

(51) Int. Cl. G01N 33/48

(52) U.S. Cl. 436/94

(57) ABSTRACT

A process of standardizing the strength of dried rumen bacterial polysaccharides is described. These dried rumen bacterial polysaccharides may then be included into a composition with vitamins, probiotics, minerals, an amino acid and/or a monosaccharide and fed to reduce the effect of diarrhea in young animals. The described mixture when used for the first few days of life is capable of reducing the severity, incidence and mortality from diarrhea. It also results in increased body weight gain.

### CROSS-REFERENCES

This application is a Divisional application of application Ser. No. 10/923,313, filed on Aug. 23, 2004, entitled “Animal Nutritional Product that Increases Weight Gain and Reduces Diarrhea Morbidity, Mortality and Severity by Stimulation of Natural Immune Response, Nutritional Support of Immune Function and Supplemental Nutricines and Probiotics.”

### FEDERALLY SPONSORED RESEARCH OR DEVELOPMENT

The development and research for this invention involved no federal or state funding. It was supported in full by private funding.

### COMPACT DISCS AND ELECTRONIC DATA

There are no electronic data or compact discs included with this submission.

### DETAILED DESCRIPTION AND SPECIFICATION

### Field of the Invention

The present invention relates to the collection, processing, sterilization and stabilizing of rumen fluid in a dry form to preserve and standardize the bacterial polysaccharides contained therein. This standardization allows the subsequent utilization of this product with a mixture of probiotics, nutricines, vitamins, minerals, an amino acid, and/or a monosaccharide. In particular this invention is fed to young animals for the first few days of life to increase weight gain, reduce diarrhea severity, morbidity and mortality by stimulation and support of the animals natural immune response.

### BACKGROUND OF THE INVENTION

Animals are raised in concentrated rearing units. These units are used on a constant basis resulting in a build up of contamination and disease organisms. The young newborn animals are frequently affected with diarrhea. Although management practices to maximize the passive immunity are used and sanitation measures followed to minimize the exposure of newborns to virulent organisms, the diarrheal disease process is the most costly disease process affecting the rearing of newborns.

There is both a political move and a public health concern with the use of antibiotics as feed additives. There are also public health concerns with the extra-label use of antibiotics in food producing animals. To maintain health and increase productivity without the use of antibiotics is the goal of many endeavors at this time (Donovan, D. C., et al, Growth and Health of Holstein Calves Fed Milk Replacers Supplemented with Antibiotics or Enteroguard, 2002, J. Dairy Sci. 85:947-950: Webb, P. R., et al, Addition of fructooligosaccharide (FOS) and sodium diacetate (SD) plus decoquinate (D) to milk replacer and starter grain fed to Holstein calves, 1992, J. Dairy Sci. Vol 75 Suppl. 1:300). As such, there are many studies and products, which attempt to increase the immuno-competence of the neonate. Vaccines, serum immunoglobulins, colostrum replacers and colostrum antibody preparations have all been used to improve the neonate's immune status.

The aim of this invention is to increase the natural local immune response by the exposure of the gut to bacterial polysaccharides in a measured, safe and controlled manner. Rumen fluid has been shown to increase growth rate in calves, decrease morbidity, mortality and use of treatments for diarrheal disease (Muscato, T. V., L. O. Tedeschi, and J. B. Russell, The Effect of Ruminal Fluid Preparations on the Growth and Health of Newborn, Milk-Fed Dairy Calves, 2002, J. Dairy Sci., 85:648-656). Rumen fluid has been shown to contain bacterial polysaccharides. These bacterial polysaccharides are considered the “active ingredient” in rumen fluid. Bacterial polysaccharides have been shown to elicit localized immunity. Rumen bacteria have been reported to have extracellular polysaccharide “coats” that are similar to those found on many Gram (−) organisms (Costerton, J. W., H. N. Damgaard and J. K. Cheng, Cell envelope morphology of rumen bacteria, 1974, J. of Bacteriology, 118:1132-1143). It is my belief that this similarity is the reason ruminal fluid bacteria are the best to use for this desired result.

We are taught in U.S. Pat. No. 6,444,210 B1 that bacterial polysaccharides have been used as vaccines to enhance specific humoral immunity and in the particular invention named they are used to enhance general cellular immunity against a wide variety of microorganisms. The mentioned patent describes a method of isolation, purification, stabilizing and using Brucella abortus and Yersinia enterocolitica outer polysaccharide as an immunizing agent. This differs from the current invention in that the current invention makes no strides toward selecting, isolating or purifying a particular polysaccharide considered effective as an immune modulator. It further differs from the current invention in that the current invention makes no effort toward selecting, isolating or purifying the bacterial polysaccharide from the rest of the ingredients in the rumen fluid, except for excluding physically large fibers and particles. Also, the number of species of bacteria in the rumen is great and there are no steps taken to reduce this number of species. Three other similar claims have been made for specific extracts of polysaccharides to be used as vaccinal agents, see U.S. Pat. Nos. 4,210,641; 6,007,818; and 6,045,805. The current invention differs from these three inventions for the aforementioned reasons.

Rumen fluid fed fresh has resulted in increased growth rate in calves, decreased morbidity, mortality and use of treatments for diarrheal disease (Muscato, T. V., L. O. Tedeschi, and J. B. Russell, The Effect of Ruminal Fluid Preparations on the Growth and Health of Newborn, Milk-Fed Dairy Calves, 2002, J. Dairy Sci., 85:648-656). The obvious problems to using fresh rumen fluid are the daily collection of the fluid. The chance of spreading disease. The need to maintain a fistulated animal on each farm. Rumen fluid may be sterilized and bottled to increase storage time. However, upon opening, the bottle must be refrigerated. Also, each farm would need to maintain the equipment to sterilize the rumen fluid. Another problem is that there is no way to accurately measure the bacterial polysaccharide content of the rumen fluid daily on the farm. It has been shown that the number of rumen bacteria are affected by time of day, diet, time following feeding, location of sampling and diet physical characteristics (Bryant, M. P., and 1. M. Robinson, Effects of Diet, Time After Feeding and Position Sampled on Numbers of Viable Bacteria in the Bovine Rumen, 1968, J. Dairy Sci., 51:1950-1955; Bryant, M. P., and I. M. Robinson, An Improved Nonselective Culture Media for Ruminal Bacteria and its use in Determining Diumal Variation in Numbers of Bacteria in the Rumen, 1961, J. Dairy Sci., 44:1446-1456). The result is a varying level of rumen bacterial polysaccharide content collected. This phenomenon was observed by other workers (Muscato, T. V., L. O. Tedeschi, and J. B. Russell, The Effect of Ruminal Fluid Preparations on the Growth and Health of Newborn, Milk-Fed Dairy Calves, 2002, J. Dairy Sci., 85:648-656).

The process of the current invention allows for the collection of rumen fluid; sterilization of the fluid to prevent disease spread; maximization of the bacterial polysaccharide in the fluid collected by proper timing of feeding and collection, and by specialized ration formulations to increase bacterial growth in the rumen; measurement of bacterial polysaccharide content; and standardization of the amount of bacterial polysaccharide used. There are patents used to measure the level of microbial activity and the presence of live organisms in fluid, however there is not a measurement to standardize the bacterial polysaccharides in a product for dosing. For examples see U.S. Pat. Nos. 5,970,163; 6,051,394; and 6,344,332 B1.

An important goal of this invention is to make the product available in an easily storable, transportable and usable form. As such this product could be administered orally to individual animals by either drenching or dosing with a solution of the product. It may be fed to individual animals by mixing it into the milk fed to that animal. It may be fed by some unique system to poultry. Or, it may be top-dressed on dry feed for swine and poultry.

To accomplish this, the rumen bacterial polysaccharides must be standardized in amount to allow mixing and dosing as needed.

### SUMMARY OF THE INVENTION

A process of standardizing the strength of dried rumen bacterial polysaccharides is described. These dried rumen bacterial polysaccharides may then be included into a composition with vitamins, probiotics, minerals, an amino acid and/or a monosaccharide and fed to reduce the effect of diarrhea in young animals. The described mixture when used for the first few days of life is capable of reducing the severity, incidence and mortality from diarrhea. It also results in increased body weight gain.

### DRAWINGS

There are no drawings.

### SPECIFICATION

The invention Method of Standardization of Bacterial Polysaccharides Found in Rumen Fluid is a method.

### PROCESS

Once the rumen fluid is removed, it should be collected into a stainless steel, glass or specially designed hard plastic receptacle to prevent any reaction between the fluid and the receptacle. The receptacle should be clean, disinfected or sterilized and rinsed with de-ionized or distilled water. This physical composition requirement and cleaning methodology will be the same for all of the numerous processing receptacles and utensils.

The fluid is then sieved through a series of sieves, starting with the largest size holes first and progressing to the smallest. The final size sieve should have holes a maximum of two (2) millimeters in diameter. The final solution will contain a slight amount of sediment. The solution should be mixed thoroughly enough to suspend this sediment and then metered into containers for autoclaving. The mixing process must be constant during filling of containers for autoclaving or the container must hold the total collection, or mixture of collections. This step is necessary to allow for testing and standardization of the bacterial polysaccharide in the final dried product.

The autoclaving process should be started immediately to prevent excess gas formation within the container that will prevent the sealing of the container. Allowing the fluid to incubate for a period of time prior to autoclaving will increase the bacterial population, however, it may also change the population (Wells, J. E. and J. B. Russell, Why Do Many Ruminal Bacteria Die and Lyse so Quickly?, 1996, J. Dairy Sci. 79:1487-1495). This probable change in population has not been studied nor the results of the resulting product tested. Although this step is contemplated and planned, until this is done, I feel that this is a major step in controlling the quality and standardization of the product.

These collections are labeled to allow control of each collected lot. Each lot collected must be tested for bacterial polysaccharides. Therefore it is important to keep each lot identified the same. In addition, each lot autoclaved must be tested for bacterial growth, both aerobic and anaerobic. It then becomes necessary that each lot autoclaved is identified, regardless of the collection lot it originated. Autoclaving should be done for a period of 45-60 minutes at of temperature of 240° F. (116° C.) and 10 pounds per square inch of pressure.

Following autoclaving, the rumen fluid collection is allowed to cool. Freeze-drying may be started immediately or it may be stored for variable amounts of time prior to further processing. Prior to freeze-drying, samples from each collection lot must be taken to be tested for bacterial polysaccharides. In addition, each lot autoclaved must be sampled and tested for bacterial growth, both aerobic and anaerobic growth. These samples must be taken prior to freeze-drying, but the testing does not have to be finished prior to freeze-drying. Each lot of freeze-dried material must be labeled with the collection lot and the autoclaved lot. The amount of material from each collection lot placed into each freeze-drying tray or lot must be recorded.

The trays are first frozen in a not frost-free freezer and then placed into the freeze-drying chamber. The freeze-drying process followed is in the following schedule. It is probable and expected that the schedule will change from one type of freeze-drying equipment to another. In addition the depth of the trays and the amount of liquid used will also affect the drying time and results.

| | | |
|---|---|---|
| Day | Set Point | Vacuum millitors |

| 1 | (−) 20° | F. | 103 |
|---|---|---|---|
| 2 | (−) 15° | F. | 104 |
| 3 | (−) 10° | F. | 106 |
| 4 | (−) 5° | F. | 99 |
| 5 | 0 | | 102 |
| 6 | 10° | F. | 87 |
| 7 | 20° | F. | 90 |
| 8 | 30° | F. | 94 |
| 9 | 40° | F. | 92 |
| 10 | 50° | F. | 91 |
| 11 | 70° | F. | 92 |

Once the product is dried, it is removed from the chamber, scraped out of the drying tray into a mixing container. At this point it is ready to be mixed into an amino acid carrier that will be used to dilute the dried rumen fluid and allow it to be standardized in the invention.

### Method

The invention Method of Standardization of Bacterial Polysaccharides Found in Rumen Fluid is a method. Without a method of standardizing the bacterial polysaccharides, there would be no way of producing a product that works consistently and remains the same from batch to batch.

Each lot collected must be tested for bacterial polysaccharides. This test will quantify the amount of bacterial polysaccharides in the lot. The amount of product that is placed into the freeze dryer may then be mathematically used to determine the amount of bacterial polysaccharides found in the lot. This dried material will then be added to an amino acid carrier to allow for standardization.

For example, if the fluid prior to drying contained 300 μg hexose equivalent bacterial polysaccharides/ml. and there were 4000 ml. dried, we simply multiply the 4000 ml.×300 μg. This would equal 1,200,000 μg of bacterial polysaccharides in the total wafer of dried material. There must be two assumptions to continue. First, how much bacterial polysaccharide is desired per dose, and second, how large is the dose of carrier material to be used? I want to use 2,400 ug of bacterial polysaccharides per dose and I would like a 5-gram inclusion per dose. This batch of dried material contains 1,200,000 ug of bacterial polysaccharides. To determine the number of doses that may be made from this batch, divide the total μg of bacterial polysaccharides by the μg of bacterial polysaccharides desired per dose. 1,200,000 μg/2,400 μg per dose=500 doses. This is the number of doses we know we can make from this mix.

From previous experiments, 4000 ml of solution will render about 80 grams of dried product. The assumption was that there would be a 5-gram inclusion per dose. It is determined that this batch will make 500 doses. 500 doses×5 grams=2,500 grams. Now, put all of the dried material into a receptacle on a scale. The receptacle should have had the tare checked prior to the dried material being placed into it. Q. S. the total with the carrier amino acid to reach 2,500 grams net weight. This material should then be thoroughly mixed. This mixed product of the carrier amino acid and the dried rumen fluid will now be standardized and may be incorporated into the rest of the mix simply by weight.

### Experimental Supporting Trials

Field trials with this mixture included with the freeze dried bacterial polysaccharide resulted in improved growth rate and weight gain over the use of the bacterial polysaccharide alone. Use of the specially collected rumen fluid bacterial polysaccharide resulted in less sick animals, less mortality and fewer treatments required in calves.

### Trials

### New Mexico Calf Treatment Trial

The objective of this study was to compare 3 different treatments for calves. The main exercise here was to find if freeze-drying was an acceptable treatment for the autoclaved rumen fluid. To ensure that each treatment was randomly assigned the treatment was assigned to the calves in the order they were delivered to the calf raiser. Both bull calves and heifer calves were treated. Each calf was assigned to the treatment group according to the order of delivery to the calf raising facility, the farm of origin and the sex of the calf. Bull calves derived from other farm(s) than C_Dairy were considered a separate subgroup. Each calf was assigned to the treatment group according to the color of the treatment that was next in the rotation. The rotation was determined to be white, green and red. There were 3 subgroups in the study: C_Dairy heifers, C_Dairy bulls and other dairies' bulls. The rotation of treatments was made within each of the subgroups. For example: Two heifers are delivered on Monday. The first is assigned to the white treatment, the second is assigned to the green treatment. The first bull delivered from C_Dairy is assigned to the White treatment. The first bull from other dairies is assigned to the White treatment. On Tuesday, four more heifers are delivered. The first is assigned to the red treatment, then white, green and red. The same type of rotation was used for C_Dairy bulls and other dairies' bulls. The C_Dairy bulls were separated from the other bulls for two reasons. First there were records available from C_Dairy on dam age and colostrum administration. Second, the other bull calves were assimilated from several other dairies and owned by the calf raisers instead of C_Dairy.

The calf raisers recorded the calf's dam's number (when available) and birth date (delivery date was considered acceptable). They also recorded which treatment the calf was assigned to. If available, they were asked to check the appropriate space if the calf was a twin or if the cow had to be helped to deliver the calf (the calf was pulled). The calf should be weighed on arrival. Colored grease markers were used to mark each pen to allow the workers the ability to quickly identify the treatment group the calves are assigned to.

The treatment assigned was given for seven days. The calves were treated only 1 time per day in the morning. The calf was to receive colostrum the first day and then receive the treatment for 7 days. The medicines used for each treatment group were:

Treatment—White Calf Treatment Group—White Powder Treatment—Freeze dried autoclaved rumen fluid with probiotics, chelated trace minerals, amino acids. Positive control—Green Calf Treatment Group—Green Liquid Treatment—Autoclaved liquid rumen fluid colored with cake coloring.

Negative Control—Red Calf Treatment Group—Red Powder Treatment—Milk powder colored with Kool-Aid®.

The mixing and feeding instructions given to the calf feeders were:

Mix the treatment in the milk prior to feeding the calf. The treatment may be mixed for several calves at once, however it may tend to settle out if allowed to stand. The bottles should be filled immediately after mixing the treatment and then inverted once or twice prior to feeding. If the milk has to stand in a five-gallon container following mixing prior to feeding or pouring into bottles, remix the container prior to pouring up for the calves. Once mixed the milk will have a color the same as the treatment group. Pink milk to the calves with a red marked pen, white milk (yellowish-gray color) to the calves with white marked pen and green milk to calves with a green marked pen.

The powder treatment is mixed at 2 level teaspoons (tsp—small spoon) per bottle. When mixing for several calves, mix Y cup rounded plus two tablespoons level per 5 gallon bucket.

The liquid treatment is mixed at the rate of 8 cc per bottle or 80 cc per 5-gallon bucket. Shake well before drawing out this treatment. A needle is not needed to draw it out of the bottle. The tops have slits that will allow a syringe tip to be inserted to facilitate drawing out the treatment.

The monitoring instructions used during this trial are as follows:

Although the treatment is only given for seven days, the effects are expected to last until weaning. The calves should be monitored daily until weaning. At weaning the calves should be weighed and the weight recorded on the sheet containing the calf's birth date and dam #.

Should any of the animals become sick, treat them, as is your normal practice and record the date and the medicaments used.

Daily—Record any calves that are sick, and the medicines administered.

Results: The weight gains were better for the treated animals in two of the trial groups. The group of heifers did not show the same response. The difference in the incoming weight of the three treatment groups within the heifer group may have contributed to this lack of response. The difference in the gain between the treatment group and the average of the two control groups as shown below is 6.3 #, 6.2 # and 0.9# respectively. Due to irregularities in the recording of illnesses and differences in the treatments used between groups (C_Dairy vs Purchased) these data were not included into the analysis.

New Mexico Calf Trial

| | Number of | | | Gain |
|---|---|---|---|---|
| | Calves in | In Weight in | Out Weight | During |
| Treatment | Group | Pounds | in Pounds | Trial |
| PBG | 17 | 88.4 | 150.6 | 61.2 |
| PBR | 15 | 93.9 | 157.9 | 64.0 |
| PBW | 17 | 90.5 | 159.4 | 68.9 |
| CBG | 17 | 90.7 | 156.8 | 66.1 |
| CBR | 17 | 89.2 | 155.4 | 66.2 |
| CBW | 17 | 88.8 | 161.2 | 72.4 |
| CHG | 17 | 77.8 | 135 | 57.2 |
| CHR | 16 | 74.8 | 134.5 | 59.7 |
| CHW | 17 | 82.0 | 141.3 | 59.3 |

P = PURCHASED
C = C_DAIRY
B = BULL CALF
H = HEIFER CALF
G = POSITIVE CONTROL with liquid product
R = NEGATIVE CONTROL
W = TREATMENT with freeze dried product

### Texas Calf Treatment Trial

The objective of this study was to compare 3 different treatments for calves. To ensure that each treatment was randomly assigned the treatment was assigned to the calves in the order they were born. Both bull calves and heifer calves were treated. Each calf was assigned to the treatment group according to the color of the card the calf's number appeared on. The cards were printed on three different color card stock. The assignment of the treatment used for each treatment group was:

Pink Calf card—Red Powder Treatment—Negative Control

White Calf card—White Powder Treatment—Warm Air Dried Positive Control

Green Calf card—Green Liquid Treatment—Treatment Group

The calf's dam's number and birth date were recorded on the cards. The workers were asked to check the appropriate space if the calf is a twin or if the cow needed assistance to deliver the calf (the calf was pulled).

The treatment assigned was given for seven days. The calves were treated only 1 time per day in the morning. The calf was to receive colostrum the first day and treatment for the next 7 consecutive days. The calf feeder was asked to circle the day of birth and then X each day the treatment is given.

The mixing and feeding instructions given to the calf feeders were:

Mix the treatment in the milk prior to feeding the calf. The treatment may be mixed for several calves at once, however it may tend to settle out if allowed to stand. The bottles should be filled immediately after mixing the treatment and then inverted once or twice prior to feeding. If the milk has to stand in a five-gallon container following mixing prior to feeding or pouring into bottles, remix the container prior to pouring up for the calves. Once mixed the milk will have a color the same as the card. Pink milk to the calves with a pink card, white milk (grayish color) to the calves with white cards and green milk to calves with a green card.

The two powder treatment are mixed as 2 level teaspoons (tsp—small spoon) per bottle. When mixing for several calves, mix 6 Tablespoons (tblsp—large spoon) per 5-gallon bucket

The liquid treatment is mixed at the rate of 8 cc per bottle or 80 cc per 5-gallon bucket Shake well before drawing out this medicine.

Although the treatment is only given for seven days, the effects are expected to last until weaning. The calves should be monitored daily until weaning or until the individual pages are collected (this may be done prior to weaning if the calves appear normal).

The monitoring instructions used during this trial are as follows:

Daily—Record the score of the manure from the calf. The scores to be used are:
1. Normal (1)—Firm but not hard. Original form is distorted slightly after dropping to floor and settling.
2. Soft (2)—Does not hold form, piles but spreads slightly. Similar to soft serve ice cream.
3. Runny (3)—Spreads readily to about Y of an inch (6 mm) in depth. Similar to pancake batter.
4. Watery (4)—Liquid consistency, splatters. Similar to orange juice.

If there is some question as to whether the manure is one score or another, for example: soft or runny, just list both scores for that day. If diarrhea develops during the day, simply write in the second score with PM after it for the later observation. If diarrhea continues for 4 days and it is watery for the four days this should be recorded each day as 4. An example of the records follows. In the example the first day (November 1) was normal and this is recorded as a 1. The second day (November 2) the calf had soft manure in the morning and watery diarrhea in the afternoon. This would be recorded as a 2 for the soft manure in the morning and as a 4 followed by PM for the watery manure in the afternoon. The next three days the calf has watery diarrhea (Nov. 3-5, and recorded as a 4). The calf is better on November 6 and the manure is not runny but really isn't firm enough to be soft. This would be recorded as a 2 for soft and a 3 for runny. On November 7 the calf is headed for recovery and the manure is soft recorded as a 2.

| Nov 1 | Nov 2 | Nov 3 | Nov 4 | Nov 5 | Nov 6 | Nov 7 |
|---|---|---|---|---|---|---|
| 1 | 2 | 4 | 4 | 4 | 2-3 | 2 |
| | 4PM | | | | | |

Treatment descriptions are:

Green Liquid Treatment—Autoclaved liquid rumen fluid

Red Powder Treatment—Milk powder with red Kool-Aid®

White Powder Treatment—Warm Air Dried Rumen Fluid on ground rice base with added probiotics, vitamins and trace minerals.

Results:

There were no differences in manure consistency scores between treatments. The number of antibiotic treatments administered to animals for diarrhea was reduced by 50% for treated calves. There were no deaths of treated calves but 4 and 3 deaths in the two control groups. No body weights were recorded in this trial.

Texas Calf Trial

| | | # antibiotic | | # animals |
|---|---|---|---|---|
| | # calves per | treatments | # | treated with |
| Treatment | treatment | for group | Deaths | antibiotics |
| GH | 12 | 9 | 0 | 6 |
| RH | 13 | 12 | 3 | 5 |
| WH | 13 | 14 | 2 | 7 |
| GB | 14 | 1 | 0 | 1 |
| RB | 13 | 8 | 1 | 5 |
| WB | 11 | 9 | 1 | 6 |
| Total G | 26 | 10 | 0 | 7 |
| Total R | 26 | 20 | 4 | 10 |
| Total W | 24 | 23 | 3 | 13 |

G = Liquid product treatment
R = Negative control
W = Heat dried positive control
B = Bull calf
H = Heifer calf
WB - Two of these calves died within 24 hours following birth

I claim:

1. A method of standardizing the dose of bacterial polysaccharides by measuring the amount of bacterial polysaccharides in the rumen fluid, measuring the amount of fluid dried, then determining the total amount of bacterial polysaccharides contained in the dried rumen fluid wafer. This quantity of bacterial polysaccharides is then used to determine the total number of doses that may be produced. The freeze dried rumen fluid wafer is then weighed and an amino acid carrier (threonine) is added to Q. S. the total to the weight represented by the weight of the total number of doses that may be produced.
